(19) United States
(12) Patent Application Publication
(10) Pub. No.: US 2026/0146844 A1
(43) Pub. Date: May 28, 2026

(54) DETECTION DEVICE

(71) Applicant: Japan Display Inc., Tokyo (JP)

(72) Inventors: Daichi ABE, Tokyo (JP); Kaoru ITO, Tokyo (JP)

(21) Appl. No.: 19/398,494

(22) Filed: Nov. 24, 2025

(30) Foreign Application Priority Data
Nov. 25, 2024 (JP) 2024-204522

(51) Int. Cl. G01B 11/06

(52) U.S. Cl. CPC G01B 11/06

(57) ABSTRACT

According to an aspect, a detection device includes: a light source part in which point light sources are arranged; a planar optical sensor in which optical sensors are arranged; an object placement member; and a control circuit. The planar optical sensor has detection areas corresponding to an arrangement of the point light sources. Each detection area includes more than one optical sensor. When predetermined conditions are satisfied, a process, in which luminance of the point light source before or after luminance change is set as adjusted luminance of the point light source, is individually performed for each combination of the point light sources and the detection areas; and the predetermined conditions are that one of two output levels of each detection area obtained before and after changing the luminance of each point light source is higher than a threshold, and that the other thereof is lower than the threshold.

### CROSS-REFERENCE TO RELATED APPLICATION

This application claims the benefit of priority from Japanese Patent Application No. 2024-204522 filed on Nov. 25, 2024, the entire contents of which are incorporated herein by reference.

### BACKGROUND

### 1. Technical Field

What is disclosed herein relates to a detection device.

### 2. Description of the Related Art

Devices are known that acquire an image by imaging a Petri dish in which a culture medium (e.g., agar) in which for culturing cultivation targets such as bacteria is formed, and detect colonies of the cultivation targets formed on the culture medium from the image (for example, Japanese Patent Application Laid-open Publication No. 2012-080802).

When a light source and an optical sensor arranged so as to face each other with the Petri dish interposed therebetween are used for imaging the Petri dish, the colonies are detected based on a change in brightness that occurs in an output of the optical sensor depending on whether the colonies are formed. However, when light from the light source is too dark or too bright, the difference in brightness is less likely to appear in the output of the optical sensor, whereby the detection of the colonies cannot be performed well in some cases.

For the foregoing reasons, there is a need for a detection device capable of better detection of colonies.

### SUMMARY

According to an aspect, a detection device includes: a light source part in which a plurality of point light sources configured to emit light are two-dimensionally arranged; a planar optical sensor in which a plurality of optical sensors configured to detect the light from the light source part are two-dimensionally arranged; an object placement member provided to allow an object to be detected to be placed between the light source part and the planar optical sensor; and a control circuit configured to control operations of the light source part and the planar optical sensor. The planar optical sensor has a plurality of detection areas corresponding to an arrangement of the point light sources. Each of the detection areas includes more than one of the optical sensors. The control circuit is configured to perform luminance adjustment of the point light sources. In the luminance adjustment: when predetermined conditions are satisfied, a process, in which luminance of the point light source before or after luminance change is set as adjusted luminance of the point light source, is individually performed for each combination of the point light sources and the detection areas corresponding to each other; and the predetermined conditions are that one of two output levels of each of the detection areas obtained before and after changing the luminance of each of the point light sources is higher than a threshold for determining an output of the detection area, and that another of the two output levels of the detection area is lower than the threshold.

### BRIEF DESCRIPTION OF THE DRAWINGS

FIG. 1 is a diagram illustrating a main configuration of a detection device;

FIG. 2 is a diagram illustrating a configuration example of an irradiated area and a wiring area;

FIG. 3 is a circuit diagram illustrating a circuit configuration of an optical sensor;

FIG. 4 illustrates schematic views illustrating configuration examples of a light source;

FIG. 5 is a schematic diagram schematically illustrating a configuration example of a detection system;

FIG. 6 is a schematic diagram illustrating a relation between one detection device and an external configuration;

FIG. 7 is a schematic view illustrating a positional relation of main components of a detection device with an object to be detected;

FIG. 8 is a plan view illustrating a concept of distinguishing positions of a plurality of point light sources by coordinates;

FIG. 9 is a plan view illustrating a concept of indicating a relation between an irradiated area and the arrangement of the point light sources by the coordinates;

FIG. 10 is a plan view schematically illustrating a relation of diffusing areas of light from the point light sources with detection areas;

FIG. 11 is a schematic view illustrating an exemplary relation of the detection areas with optical sensors;

FIG. 12 illustrates schematic views illustrating an example of a process to sequentially determine an intensity of light emitted from each of the point light sources;

FIG. 13 is a graph schematically illustrating steps of controlling strength and weakness of the light from the point light source;

FIG. 14 is a timing diagram schematically illustrating a process of gradual reduction of luminance levels of point light sources by an automatic luminance adjustment performed in an embodiment of the present disclosure;

FIG. 15 is a flowchart of processing related to operations of the detection device;

FIG. 16 is a flowchart of an initial process;

FIG. 17 is a flowchart of a periodic operation;

FIG. 18 illustrates schematic views illustrating another example of the process to sequentially determine the intensity of the light emitted from each of the point light sources; and

FIG. 19 is a timing diagram schematically illustrating a process of gradual increase of the luminance levels of the point light sources by the automatic luminance adjustment performed in a modification of the embodiment.

### DETAILED DESCRIPTION

The following describes an embodiment of the present disclosure with reference to the drawings. What is disclosed herein is merely an example, and the present disclosure naturally encompasses appropriate modifications easily conceivable by those skilled in the art while maintaining the gist of the present invention. To further clarify the description, the drawings may schematically illustrate, for example, widths, thicknesses, and shapes of various parts as compared with actual aspects thereof. However, they are merely examples, and interpretation of the present disclosure is not limited thereto. The same element as that illustrated in a drawing and the present specification that has already been discussed is denoted by the same reference numeral through the description and the drawings, and detailed description thereof may not be repeated where appropriate.

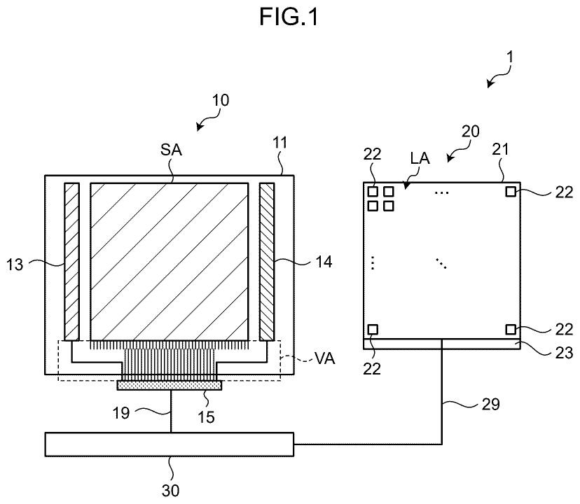

FIG. 1 is a diagram illustrating a main configuration of a detection device 1. The detection device 1 includes a planar optical sensor 10, a light source panel 20, and a control circuit 30. The planar optical sensor 10 and the light source panel 20 of the detection device 1 are coupled to the control circuit 30.

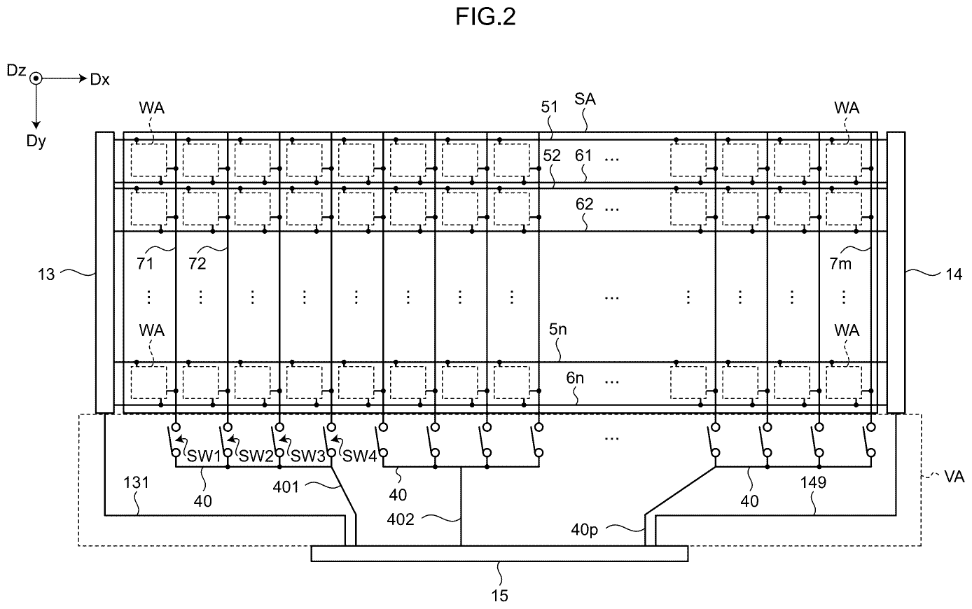

The planar optical sensor 10 is provided with an irradiated area SA (refer to FIG. 2) on a substrate 11. A reset circuit 13, a scan circuit 14, and a wiring area VA are provided on the substrate 11. Components on the irradiated area SA, the reset circuit 13, and the scan circuit 14 are coupled to a detection circuit 15 via the wiring area VA.

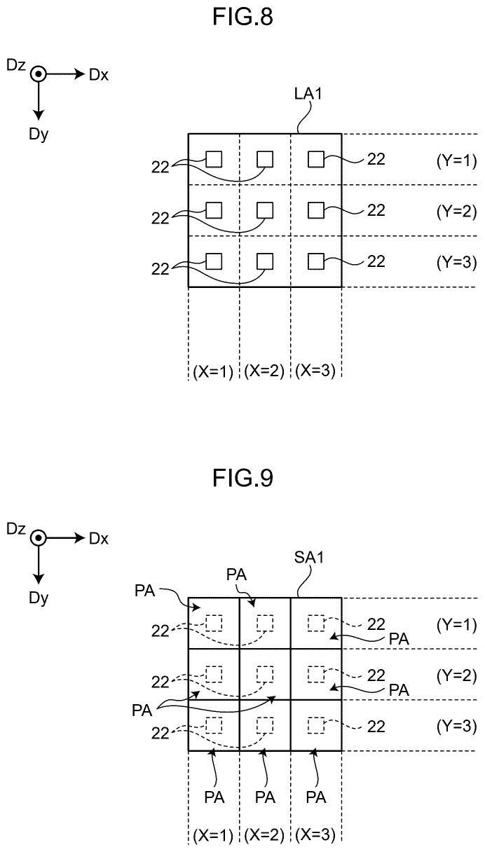

The light source panel 20 has a light-emitting area LA that emits light to the irradiated area SA. The light source panel 20 is provided with a point light source 22 on a substrate 21. The point light source 22 emits light. Specifically, the point light source 22 includes a light-emitting element, such as a light-emitting diode (LED), that serves as the point light source, and is provided in the light-emitting area LA. In the example illustrated in FIG. 1 and in FIGS. 8 to 10 to be explained later, a plurality of the point light sources 22 are arranged in a matrix having a row-column configuration along a first direction Dx and a second direction Dy on the substrate 21. Thus, the light source panel 20 corresponds to a light source part in which the point light sources 22 emitting the light are two-dimensionally arranged.

The light source panel 20 is provided with a light source drive circuit 23. Under the control of the control circuit 30, the light source drive circuit 23 controls turning on and off of each of the point light sources 22 and the luminance thereof when being turned on. The point light sources 22 may be provided so as to be individually controllable in light emission, or may be provided so as to emit light all together.

The control circuit 30 performs various processes related to operations of the detection device 1. Specifically, the control circuit 30 is a circuit, such as a field-programmable gate array (FPGA) that can implement a plurality of functions. The control circuit 30 may have other configurations, such as an application-specific integrated circuit (ASIC). The control circuit 30 is coupled to the light source drive circuit 23 via wiring 29 and performs processing related to the lighting of the point light sources 22, such as determination of lighting patterns and lighting timings of the point light sources 22.

The control circuit 30 is coupled to the detection circuit 15 via wiring 19 and obtains an output from the detection circuit 15. The control circuit 30 also controls the timing of obtaining the output from the detection circuit 15, that is, the timing of operating the scan circuit 14 so as to provide a gate signal to a scan line 6. Thus, the control circuit 30 controls operations of the point light sources 22 and the planar optical sensor 10. The control circuit 30 further performs processing based on outputs of a plurality of optical sensors WA. Such processing includes a determination process to determine whether colonies have been formed.

Although not illustrated in the drawings, the detection device 1 includes an analog-to-digital conversion circuit, a digital-to-analog conversion circuit, and other components. The analog-to-digital conversion circuit is a circuit for allowing the outputs from the optical sensors WA (refer to FIG. 2) transmitted via the detection circuit 15 to be handled in arithmetic processing by the control circuit 30. The digital-to-analog conversion circuit is a circuit for allowing digital signals generated by the arithmetic processing of the control circuit 30 to be used for controlling the operations of the planar optical sensor 10 and the light source panel 20. These circuits may be included, for example, in part or in whole in the detection circuit 15. These circuits may alternatively be functions performed by circuits mounted on flexible printed circuits (FPCs) provided as the wiring 19 and the wiring 29. These circuits may alternatively be mounted in other ways on the detection device 1.

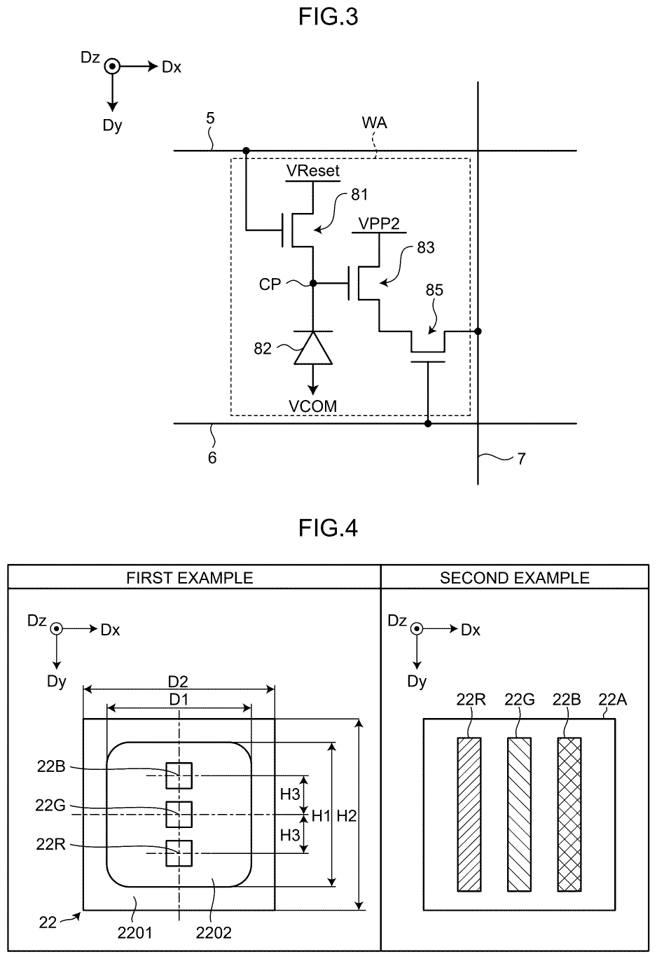

FIG. 2 is a diagram illustrating a configuration example of the irradiated area SA and the wiring area VA. The optical sensors WA (FIG. 3) are two-dimensionally arranged in the irradiated area SA of the planar optical sensor 10. In the embodiment, as illustrated in FIG. 2, the optical sensors WA are arranged in a matrix having a row-column configuration along the first direction Dx and the second direction Dy. The first direction Dx is orthogonal to the second direction Dy. In the following description, the term “third direction Dz” refers to a direction orthogonal to the first direction Dx and the second direction Dy.

The reset circuit 13 is coupled to reset signal transmission lines 51, 52, . . . , 5n. Hereinafter, the term “reset signal transmission line 5” refers to any one of the reset signal transmission lines 51, 52, . . . , 5n. The reset signal transmission line 5 is wiring along the first direction Dx. In the example illustrated in FIG. 2, n reset signal transmission lines 5 are arranged in the second direction Dy. n is a natural number equal to or larger than 2. The n reset signal transmission lines 5 are each coupled, at one end in the first direction Dx, to the reset circuit 13.

The scan circuit 14 is coupled to scan lines 61, 62, . . . , 6n. Hereinafter, the term “scan line 6” refers to any one of the scan lines 61, 62, . . . , 6n. The scan line 6 is wiring along the first direction Dx. In the example illustrated in FIG. 2, n scan lines 6 are arranged in the second direction Dy. The n scan lines 6 are each coupled, at the other end in the first direction Dx, to the scan circuit 14.

As illustrated in FIG. 2, the reset signal transmission lines 5 and the scan lines 6 are alternately arranged in the second direction Dy in the irradiated area SA. The reset circuit 13 and the scan circuit 14 illustrated in FIGS. 1 and 2 are arranged at locations facing each other with the irradiated area SA interposed therebetween, but the layout of the reset circuit 13 and the scan circuit 14 is not limited to this layout and can be changed as appropriate.

Signal lines 71, 72, . . . , 7m are provided in the irradiated area SA. Hereinafter, the term “signal line 7” refers to any one of the signal lines 71, 72, . . . , 7m. The signal line 7 is wiring along the second direction Dy.

In the example illustrated in FIG. 2, m signal lines 7 are arranged in the first direction Dx. m is a natural number equal to or larger than 2. The m signal lines 7 are each coupled, at one end in the second direction Dy, to one of a plurality of switches (for example, a switch SW1, a switch SW2, a switch SW3, or a switch SW4) included in a multiplexer 40.

The multiplexer 40 is provided in the wiring area VA. The multiplexer 40 includes a plurality of switches. In the example illustrated in FIG. 2, the switches SW1, SW2, SW3, and SW4 are illustrated as the switches. The switches included in one multiplexer 40 are turned on (conducting state) at different times from one another. During a period when one of the switches included in one multiplexer 40 is on (conducting state), the other switches are off (non-conducting state). The number of the multiplexers 40 corresponds to the number (m) of the signal lines 7. When the number of the switches is p, m/p is sufficient as the number of the multiplexers 40. When more than one multiplexer 40 are provided, each of the multiplexers 40 is coupled to the detection circuit 15 via an individual one of wiring lines 401, 402, . . . , 40p.

The coupling between the signal lines 7 and the detection circuit 15 via the multiplexer 40 is merely exemplary and is not limited to this example. The signal lines 7 may be individually directly coupled to the detection circuit 15 in the wiring area VA. In the wiring area VA, the reset circuit 13 is coupled to the detection circuit 15 via wiring 131. In the wiring area VA, the scan circuit 14 is coupled to the detection circuit 15 via wiring 149.

In the detection of light by a photodiode (PD) 82 (refer to FIG. 3) provided in the optical sensor WA, the detection circuit 15 outputs signals to control operation timing of the reset circuit 13 and the scan circuit 14 under the control of the control circuit 30. The detection circuit 15 receives the outputs from the optical sensors WA. The detection circuit 15 converts the signals received from the optical sensors WA into data that can be interpreted by the control circuit 30 and outputs the data to the control circuit 30. The detection circuit 15 of the embodiment is a microcontroller unit (MCU).

FIG. 3 is a circuit diagram illustrating a circuit configuration of the optical sensor WA. The first direction Dx and the second direction Dy in FIG. 3 merely correspond to the directions of the reset signal transmission lines 5, the scan lines 6, and the signal lines 7, and do not exactly indicate the relative positional relation of the circuit configuration in the optical sensor WA.

As illustrated in FIG. 3, a switching element 81, the PD 82, a transistor element 83, and a switching element 85 are provided in the optical sensor WA. The PD 82 is a photodiode (PD). The switching elements 81 and 85 and the transistor element are metal-oxide semiconductor field-effect transistors (MOSFETs).

The gate of the switching element 81 is coupled to the reset signal transmission line 5. One of the source and the drain of the switching element 81 is supplied with a reset potential VReset. The other of the source and the drain of the switching element 81 is coupled to the cathode of the PD 82 and the gate of transistor element 83. Hereinafter, the term “coupling part CP” refers to a point where the other of the source and the drain of the switching element 81 is coupled to the cathode of the PD 82 and the gate of transistor element 83. A reference potential VCOM is supplied from the anode side of the PD 82. The potential difference between the reset potential VReset and the reference potential VCOM is set in advance, but the reset potential VReset and the reference potential VCOM may be variable. The reset potential VReset is higher than the reference potential VCOM.

The drain of the transistor element 83 serving as a source follower is supplied with an output source potential VPP2. The source of the transistor element 83 is coupled to one of the source and the drain of the switching element 85. The other of the source and the drain of the switching element 85 is coupled to the signal line 7. The gate of the switching element 85 is coupled to the scan line 6.

The reset potential VReset, the reference potential VCOM, and the output source potential VPP2 are supplied by the detection circuit 15 to the optical sensor WA based on, for example, electric power supplied via a power supply circuit (not illustrated) coupled to the detection circuit 15. The output form of these potentials is not limited to this form, and can be changed as appropriate.

The output source potential VPP2 is set in advance. The potential on the source side of the transistor element 83 is a potential lower than the output potential of the PD 82 by a voltage (Vth) between the gate and the source of the transistor element 83. In this case, the potential on the source side of the transistor element 83 corresponds to the reset potential VReset and the reference potential VCOM. The potential of the output of the PD 82 corresponds to photovoltaic power generated by the PD 82 in response to the light detected by the PD 82 during an exposure period.

When the gate of the switching element 85 is turned on by the gate signal supplied from the scan circuit 14 via the scan line 6, the source and the drain of the switching element 85 are brought into a conducting state therebetween. This operation transmits, to the signal line 7 via the switching element 85, a signal (potential) transmitted via the transistor element 83 to the switching element 85. Thus, the output from the optical sensor WA is generated. Hereinafter, the term “gate signal” refers to the signal (potential) supplied from the scan circuit 14 via the scan line 6. The scan circuit 14 is a circuit that outputs the gate signal. As described with reference to FIGS. 2 and 3, the optical sensors WA coupled to the scan lines 6 and the signal lines 7 are arranged in a matrix having a row-column configuration in the irradiated area SA of the planar optical sensor 10. The scan line 6 is provided along the first direction Dx and is configured to transmit the gate signal that causes the optical sensors WA to generate the outputs. The signal line 7 is configured to transmit the outputs of the optical sensors WA along the second direction Dy.

The output of one PD 82 provided in one optical sensor WA corresponds to the intensity of the light detected by the PD 82 during the exposure period set in advance. The output of the PD 82 is reset in response to a signal supplied by the reset circuit 13 via the reset signal transmission line 5. When the signal turns on the gate of the switching element 81, the source and the drain of the switching element 81 are brought into a conducting state therebetween. This operation resets the potential of the coupling part CP to the reset potential VReset.

FIG. 4 illustrates schematic views illustrating configuration examples of the point light source 22. As illustrated in FIG. 4, the point light source 22 includes a first light source 22R, a second light source 22G, and a third light source 22B. The first light source 22R, the second light source 22G, and the third light source 22B are point light sources (such as LEDs) that emit light in different colors from one another. In the embodiment, the first light source 22R emits red (R) light. The second light source 22G emits green (G) light. The third light source 22B emits blue (B) light.

As illustrated as “First Example” in FIG. 4, the point light source 22 has, for example, a light-emitting area 2202 and a frame area 2201. In the light-emitting area 2202, the first light source 22R, the second light source 22G, and the third light source 22B are arranged along the second direction Dy as viewed from a planar viewpoint. The frame area 2201 is a frame-like area surrounding the light-emitting area 2202. A width D1 in the first direction Dx of the light-emitting area 2202 is smaller than a width D2 in the first direction Dx of the frame area 2201. A height H1 in the second direction Dy of the light-emitting area 2202 is smaller than a height H2 in the second direction Dy of the frame area 2201. The distance between the first light source 22R and the second light source 22G is a distance H3. The distance between the second light source 22G and the third light source 22B is also the distance H3. The distance H3 is less than half the height H1. In “First Example” in FIG. 4, the width D1 is equal to the height H1, and the width D2 is equal to the height H2, but at least one of the widths may differ from a corresponding one of the heights. The point light source 22 may be replaced with a light source of another form, specifically, such as a light source 22A illustrated in “Second Example” in FIG. 4. In the light source 22A, the longitudinal directions of the first light source 22R, the second light source 22G, and the third light source 22B are along the second direction Dy, and the first light source 22R, the second light source 22G, and the third light source 22B are arranged in this order from one side toward the other side in the first direction Dx. First Example and Second Example in FIG. 4 are exemplary forms of the light source according to the present disclosure, which is not limited to these examples. For example, the shape of the first light source 22R, the second light source 22G, and the third light source 22B in the point light source 22 as viewed from a planar viewpoint and the positional relation between the first light source 22R, the second light source 22G, and the third light source 22B can be changed as appropriate. The term “planar viewpoint” refers to a viewpoint from which a plane along the first direction Dx and the second direction Dy (Dx-Dy plane) is squarely viewed.

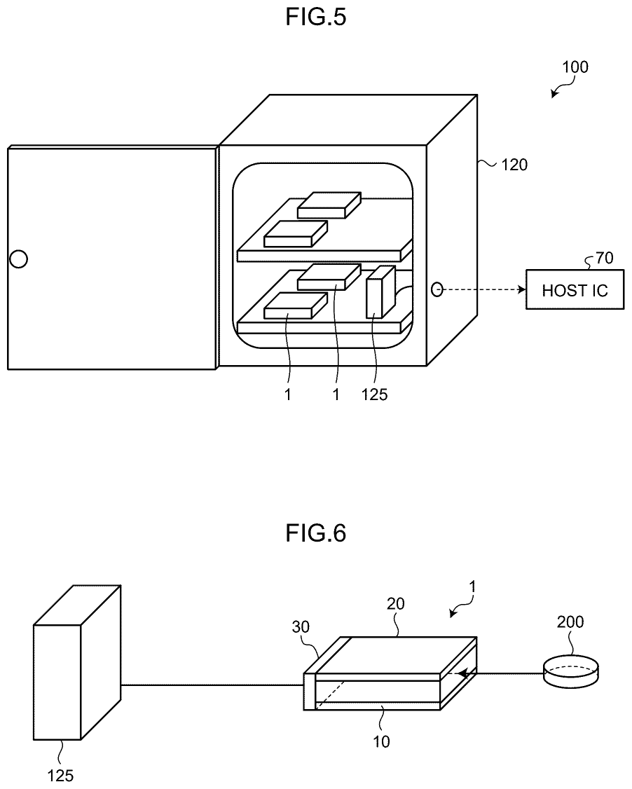

FIG. 5 is a schematic diagram schematically illustrating a configuration example of a detection system 100 including the detection device 1. As illustrated in FIG. 5, the detection system 100 includes a plurality of the detection devices 1, a host integrated circuit (IC) 70, and a coupling circuit 125. The detection devices 1 are electrically coupled to the common host IC 70 via the coupling circuit 125. The host IC 70 may be located in an incubator 120.

The incubator 120 illustrated in FIG. 5 is maintained such that an environment (temperature, humidity, and the like) therein is suitable for cultivation at an object to be detected 200 while a door is closed. The detection devices 1 are placed in the incubator 120.

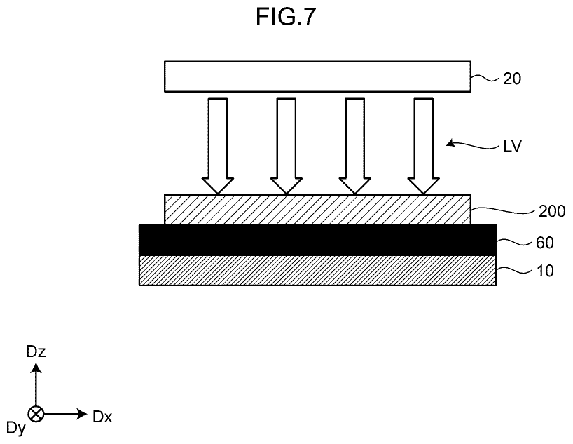

FIG. 6 is a schematic diagram illustrating a relation between one of the detection devices 1 and an external configuration. As illustrated in FIG. 6, the detection device 1 is coupled to the coupling circuit 125 by coupling the control circuit 30 to the coupling circuit 125. As illustrated in FIG. 6 and FIG. 7, which is to be described later, the planar optical sensor 10 faces the light source panel 20. The object to be detected 200 can be placed between the planar optical sensor 10 and the light source panel 20.

FIG. 6 only schematically illustrates a rough relation between the planar optical sensor 10, the light source panel 20, and the object to be detected 200. A specific structure for placing the object to be detected 200 between the planar optical sensor 10 and the light source panel 20 will be described with reference to FIG. 7.

FIG. 7 is a schematic view illustrating a positional relation of main components of the detection device 1 with the object to be detected 200. When the object to be detected 200 is placed between the planar optical sensor 10 and the light source panel 20, the object to be detected 200 is placed on a member 60, for example, as illustrated in FIG. 7. The member 60 serves as an object placement member that is provided to allow the object to be detected 200 to be positioned between the irradiated area SA and the light source panel 20.

In the embodiment, the planar optical sensor 10 is located below the object to be detected 200, and the light source panel 20 is located above the object to be detected. The member 60 of the embodiment also serves as an optical member that limits the light that is emitted from the point light source 22 of the light source panel 20 and reaches the planar optical sensor 10. Specifically, the member 60 includes any one of a plate-shaped louver, a cylindrical opening, and a microlens. The plate-shaped louver has a plurality of plate-like structures arranged in parallel and having plate surfaces along the third direction Dz. The structures are preferably made of a material having a strong light-absorbing property. The member 26 is provided along a plane (Dx-Dy plane) orthogonal to the third direction Dz. The cylindrical opening penetrates the member 60 in the third direction Dz with respect to the base of the member 60. The base is preferably made of a material having a strong light-absorbing property. The microlens is a small lens having an optical axis along the third direction Dz. The base of the member 60 that supports the microlens is preferably made of a material having a strong light-absorbing property. The member 60 as the optical member is provided in order to limit the traveling direction of the light emitted from the point light source 22 and reaching the planar optical sensor 10 to the third direction Dz or a direction having a shallower inclination angle with respect to the third direction Dz.

In the embodiment, the member 60 serves as both an optical member and a member provided so that the object to be detected 200 can be placed thereon. The optical member and the member provided so that the object to be detected 200 can be placed thereon may be provided separately from each other. For example, the member provided so that the object to be detected 200 can be placed thereon may be a plate-like member provided with a hole capable of accommodating therein the object to be detected 200. The arrangement of the light source panel 20 and the planar optical sensor 10 may be reversed. In that case, the member 60 is located, for example, on the upper side of the object to be detected 200 so as to be positioned between the object to be detected 200 and the planar optical sensor 10.

The object to be detected 200 is a culture medium (e.g., agar) accommodated in a dish of a container. The dish is specifically a Petri dish. The culture medium is a culture medium capable of culturing colonies. Hereinafter, the term simply called “colonies” refers to colonies of cultivation targets that have been cultured on/in the culture medium formed on the object to be detected 200. The cultivation targets are objects, such as biological tissues or microorganisms, that are assumed to be cultured in the culture medium. The culture medium has a light-transmitting property such that the degree of light transmission changes depending on the presence or absence of the colonies and the thickness of the colonies. A light-transmitting lid covering the dish may be further provided.

The following describes processing related to control of the intensity of the light emitted from the point light sources 22. As a premise of the following description, a relation of the point light sources 22 arranged in the light-emitting area LA with the irradiated area SA will be described with reference to FIGS. 8 to 11.

FIG. 8 is a plan view illustrating a concept of distinguishing positions of the point light sources 22 by coordinates. A light-emitting area LA1 illustrated in FIG. 8 is, for example, a part of the light-emitting area LA, but may be the whole light-emitting area LA. In the light-emitting area LA1 illustrated in FIG. 8, three rows each of which includes three point light sources 22 arranged in the first direction Dx are arranged in the second direction Dy. That is, 3×3=9 point light sources 22 are disposed in the light-emitting area LA1.

In FIG. 8, different coordinates in the first direction Dx are assigned to partial areas in the light-emitting area LA1 in each of which the three point light sources 22 arranged in the first direction Dx are arranged. The coordinates in the first direction Dx, herein, can be expressed in the form of (X=α), such as (X=1), (X=2), and (X=3). In FIG. 8, different coordinates in the second direction Dy are assigned to partial areas in the light-emitting area LA1 in each of which the three point light sources 22 arranged in the second direction Dy are arranged. The coordinates in the second direction Dy, herein, can be expressed in the form of (Y=β), such as (Y=1), (Y=2), and (Y=3). That is, the nine point light sources 22 illustrated in FIG. 8 can each be expressed in the form of (X, Y)=(α, β) as a combination of a coordinate in the first direction Dx and a coordinate in the second direction Dy. For example, the coordinates of one of the nine point light sources 22 illustrated in FIG. 8 that is located in the center can be expressed as (X, Y)=(2, 2).

FIG. 9 is a plan view illustrating a concept of indicating the relation between an irradiated area SA1 and the arrangement of the point light sources 22 by the coordinates. The irradiated area SA1 illustrated in FIG. 9 is a portion of the irradiated area SA that overlaps the light-emitting area LA1 (refer to FIG. 8) as viewed from a planar viewpoint. Detection areas PA are portions included in the irradiated area SA1 and each receive light mainly from a corresponding one of the point light sources 22 described with reference to FIG. 8. Each of the detection area PA can be indicated by a combination of the coordinate in the first direction Dx and the coordinate in the second direction Dy described with reference to FIG. 8. More specifically, by delimiting the irradiated area SA1 with a center line halving the interval between the adjacent point light sources 22, each portion of the irradiated area SA1 can be expressed in the form (X, Y)=(α, β) as illustrated in FIG. 9. For example, the detection area PA at (X, Y)=(2, 2) illustrated in FIG. 9 is a portion of the irradiated area SA1 and is a portion overlapping the point light source 22 at (X, Y)=(2, 2) illustrated in FIG. 8.

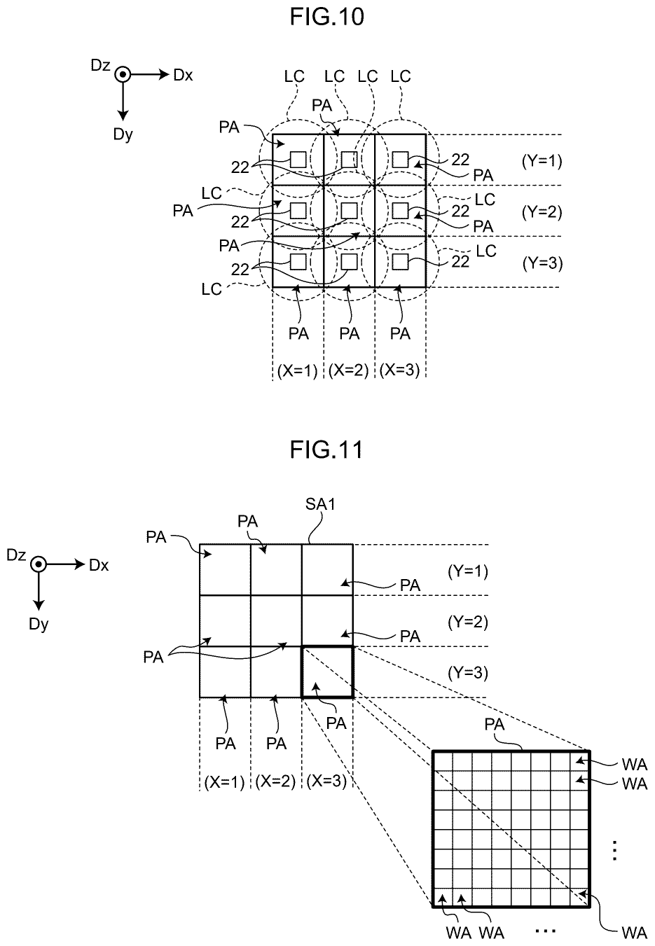

FIG. 10 is a plan view schematically illustrating a relation of diffusing areas of the light from the point light sources 22 with the detection areas PA. In FIG. 10, a diffusing area of light from one point light source 22 when having reached the irradiated area SA1 is illustrated as one area LC centered on the one point light source 22. Thus, in FIG. 10, nine areas LC corresponding to the nine point light sources 22 are illustrated.

One of the areas LC that represents the diffusing area of the light from the one point light source 22 located at (X, Y)=(α, β) covers the detection area PA at (X, Y)=(α, β) as viewed from a planar viewpoint. The one of the areas LC extends to portions of the other detection areas PA adjacent to the detection area PA. That is, the intensity of the light irradiating each of the detection areas PA is affected by the multiple point light sources 22.

The number of the point light sources 22 that affect the intensity of the light irradiating each of the detection areas PA depends on the location of the detection area PA. Specifically, the detection area PA adjacent to a larger number of the detection areas PA in the first direction Dx or the second direction Dy is affected by light from a larger number of the point light sources 22.

For example, the detection area PA at (X, Y)=(2, 2) is adjacent to a total of four detection areas PA at (X, Y)=(1, 2), (2, 1), (2, 3), and (3, 2). Therefore, the intensity of the light that irradiates the detection area PA at (X, Y)=(2, 2) is affected by light from a total of four point light sources 22 at (X, Y)=(1, 2), (2, 1), (2, 3), and (3, 2) in addition to light from the point light source 22 at (X, Y)=(2, 2).

The detection area PA at (1, 2) is adjacent to a total of three detection areas PA at (X, Y)=(1, 1), (1, 3), and (2, 2). Therefore, the intensity of the light that irradiates the detection area PA at (X, Y)=(1, 2) is affected by light from a total of three point light sources 22 at (X, Y)=(1, 1), (1, 3), and (2, 2) in addition to the light from the point light source 22 at (X, Y)=(1, 2). In the same way, each of the detection areas PA at (X, Y)=(2, 1), (2, 3), and (3, 2) is affected by light from the point light sources 22 at three pairs of coordinates adjacent to the coordinates of the detection area PA, in addition to light from the point light source 22 at the same coordinates as the detection area PA.

The detection area PA at (1, 1) is adjacent to a total of two detection areas PA at (X, Y)=(1, 2) and (2, 1). Therefore, the intensity of the light irradiating the detection area PA at (X, Y)=(1, 1) is affected by light from a total of two point light sources 22 at (X, Y)=(1, 2) and (2, 1), in addition to the light from the point light source 22 at (X, Y)=(1, 1). In the same way, each of the detection areas PA at (X, Y)=(1, 3), (3, 1), and (3, 3) is affected by light from the point light sources 22 at two pairs of coordinates adjacent to the coordinates of the detection area PA, in addition to light from the point light source 22 at the same coordinates as the detection area PA.

Light from the point light source 22 at coordinates adjacent to the coordinates of one detection area PA in a diagonal direction that intersects the first direction Dx and the second direction Dy may also affect the one detection area PA. The number of the point light sources 22 adjacent to the one detection area PA in the diagonal direction also increases as the number of the detection areas PA adjacent thereto in the first direction Dx or the second direction Dy increases. Therefore, the intensity of the light irradiating the detection area PA that has four adjacent detection areas PA in the first direction Dx or the second direction Dy is stronger than the intensity of the light irradiating the detection area PA that has three or fewer adjacent detection areas PA in the first direction Dx or the second direction Dy. The intensity of the light irradiating the detection area PA that has three adjacent detection areas PA in the first direction Dx or the second direction Dy is stronger than the intensity of the light irradiating the detection area PA that has two adjacent detection areas PA in the first direction Dx and the second direction Dy.

A plurality of the optical sensors WA are arranged in one detection area PA. In the embodiment, the intensity of the light irradiating one detection area PA is the average of the light intensities detected by the respective optical sensors WA arranged in the one detection area PA.

FIG. 11 is a schematic view illustrating an exemplary relation of the detection area PA with the optical sensors WA. While FIG. 11 exemplarily illustrates the detection area PA at (X, Y)=(3, 3) in a magnified way, the detection area PA at other coordinates is also the same as the detection area PA at (X, Y)=(3, 3).

In the embodiment, a plurality of the optical sensors WA are arranged in one detection area PA. In the detection area PA illustrated in FIG. 11, eight optical sensors WA are arranged in the first direction Dx in a row, and eight rows of the optical sensors WA are arranged in the second direction Dy. That is, 8×8=64 optical sensors WA are arranged in the detection area PA. This is merely an example. The optical sensors WA arranged in one detection area PA are limited to this example. The number of the optical sensors WA arranged in one detection area PA may be changed as appropriate.

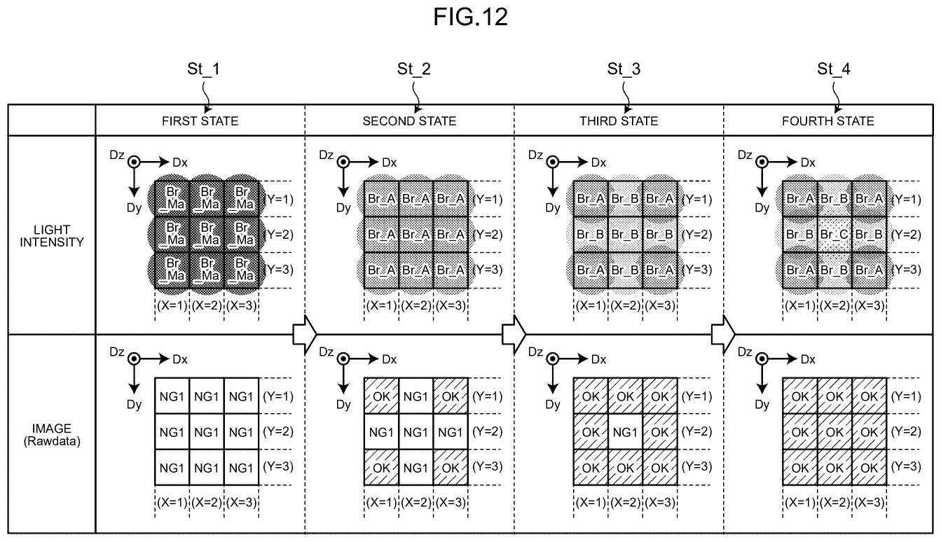

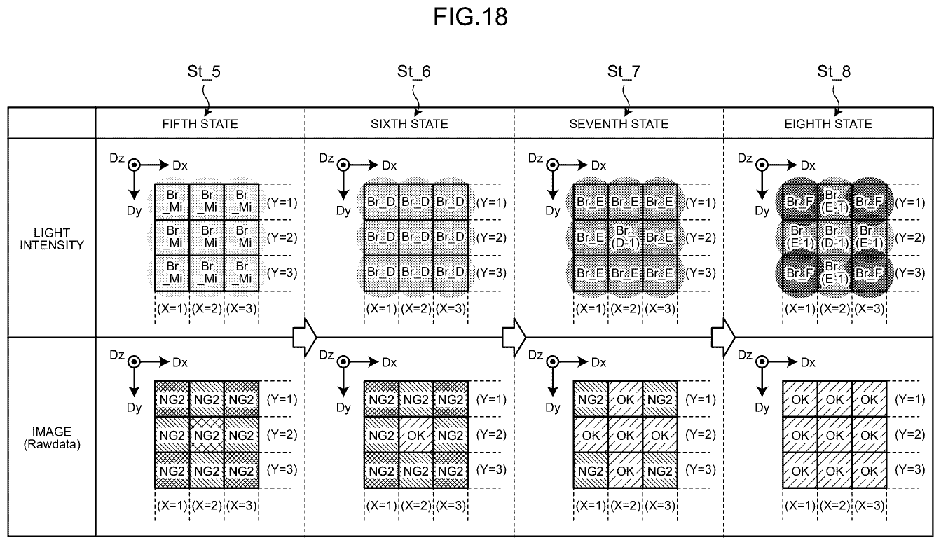

In FIGS. 8 to 11 and in FIGS. 12 and 18 to be explained later, the light-emitting area LA1 where the nine point light sources 22 are arranged and the irradiated area SA1 including nine detection areas PA are described as examples. However, the value of each of α and β in (X, Y)=(α, β) is not limited to any of the natural numbers from 1 to 3. For example, when the point light sources 22 and the optical sensors WA are arranged in a 1 to 1 relation, a may be any of the natural numbers from 1 to m (refer to FIG. 2). B may be any of the natural numbers from 1 to n (refer to FIG. 2). When the point light sources 22 and the optical sensors WA are arranged in a relation of 1:q², α may be any of the natural numbers from 1 to m/q. β may be any of the natural numbers from 1 to n/q.

The following describes the processing related to the control of the intensity of the light emitted from the point light sources 22 in the embodiment, with reference to FIGS. 12 to 14. Each of the point light sources 22 is individually controlled in terms of the intensity of the light emitted therefrom.

FIG. 12 illustrates schematic views illustrating an example of a process to sequentially determine the intensity of the light emitted from each of the point light sources 22. The term “light intensity” in FIG. 12 schematically indicates the intensity of the light emitted from each of the point light sources 22. The term “image (Rawdata)” in FIG. 12 individually indicates the acceptability of the light intensity based on an output of the irradiated area SA1 for each of the detection areas PA. The image herein refers to an image in which an output of one optical sensor WA is considered to be one pixel and a plurality of the pixels are arranged so as to correspond to the arrangement of the optical sensors WA in the irradiated area SA. Hereinafter, the term simply called “image” refers to the set of the outputs of the optical sensors WA, unless otherwise noted. The term simply called “pixel” refers to the output of the optical sensor WA, unless otherwise noted. In practice, a process such as an analog-to-digital conversion is performed to regard the output of the optical sensor WA as the pixel. This process is performed by the detection circuit 15 in the embodiment, as described above, but may be performed by the control circuit 30.

The planar optical sensor 10 is configured to output data reflecting the intensity of the light rays that have been emitted from the point light sources 22 and reached the optical sensors WA through the object to be detected 200. The data herein is data based on a set of the outputs from the optical sensors WA and can be regarded as data of an image. The term “Rawdata” indicates an output of the detection area PA in a state in which no special image processing has been applied (unprocessed).

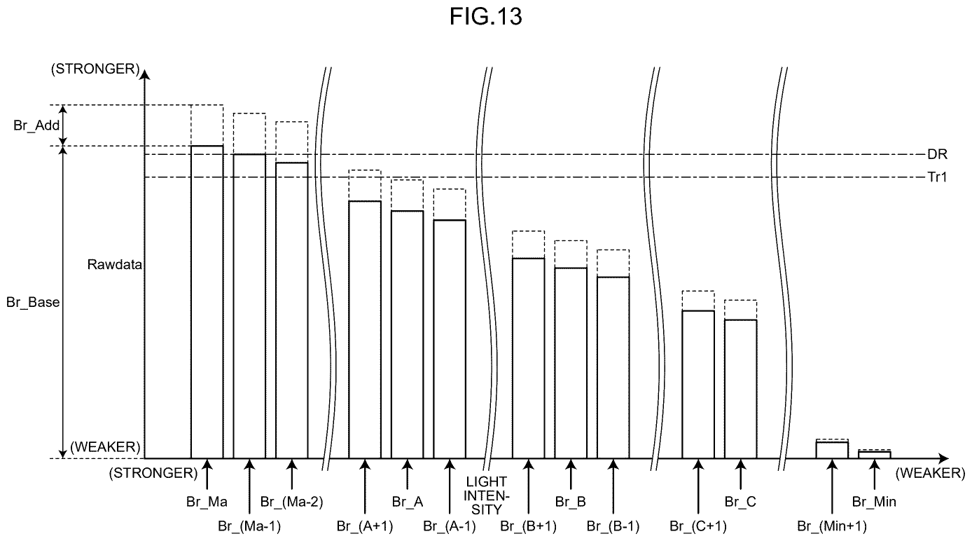

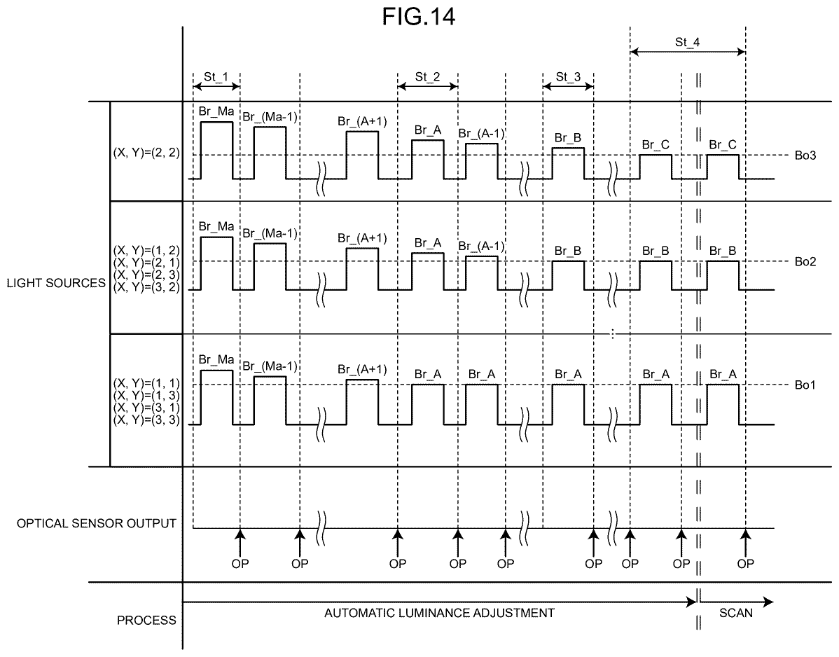

A first state St_1 illustrated in FIG. 12 is a state in which the point light sources 22 are all lit at the highest luminance. The highest luminance herein may be, but is not required to be, the highest luminance in the technical specification that can be achieved by the point light source 22. The highest luminance may be, for example, the highest luminance within an “adjustable luminance range of the point light source 22” that is preset in the operation of the detection device 1, and this highest luminance may be lower than the highest luminance in the technical specification that can be achieved by the point light source 22. In FIG. 12 and in FIGS. 13 and 14 to be explained later, the intensity of light corresponding to the luminance “Br_Ma” indicates that the luminance of the point light source 22 is the highest. A circle with a dot pattern marked with “Br_Ma” in FIG. 12 is the area LC that has been described with reference to FIG. 10. A circle with a dot pattern marked with any one of “Br_A”, “Br_B”, “Br_C”, “Br_Min”, “Br_D”, “Br_E”, and “Br_F” is also the area LC that has been described with reference to FIG. 10. That is, in FIG. 12 and in FIG. 18 to be explained later, the intensity of the light emitted from the point light source 22 is indicated by the sign added to the circle with the dot pattern indicating the area LC.

As described with reference to FIG. 7, in the embodiment, the object to be detected 200 is placed between the planar optical sensor 10 and the light source panel 20. In this state, the point light sources 22 of the light source panel 20 emit the light, and the light that has passed through the object to be detected 200 and the member 60 is detected by the planar optical sensor 10, so that the data of the image is obtained. The intensity of the light indicated by Rawdata preferably exhibits a bright/dark difference depending on whether colonies have been formed on the culture medium of the object to be detected 200. This is because the degree of transmission of light differs between the culture medium with no colonies formed thereon and the culture medium with colonies formed thereon. In other words, the detection device 1 is provided so as to be capable of using the bright/dark difference to determine whether colonies have been formed. However, if the light from the point light source 22 is too strong, the bright/dark difference may not appear in the image data.

FIG. 13 is a graph schematically illustrating steps of controlling strength and weakness of the light from the point light source 22. The horizontal axis of the graph in FIG. 13 indicates the intensity of the light from each of the point light sources 22. The vertical axis of the graph in FIG. 13 indicates the height of output level of Rawdata indicating the light intensity detected by each of the detection areas PA. Thus, each of the bar graphs in FIG. 13 indicates the output level of Rawdata output according to the intensity of light emitted from the point light source 22. The height of each of the bar graphs in FIG. 13 is illustrated ignoring an upper limit imposed by a dynamic range to be described later.

Each of the detection areas PA is affected by the intensity of the light emitted from one point light source 22 located at the same coordinates as those of the detection area PA and the intensity of the light emitted from another point light source 22 located at the coordinates of a detection area PA adjacent to the detection area PA. That is, the output level of Rawdata reflects the intensity of the light emitted from one point light source 22 located at the same coordinates as those of the detection area PA and the intensity of the light emitted from another point light source 22 located at the coordinates of a detection area PA adjacent to the detection area PA.

In FIG. 13, “Br_Base” indicates a component of the output level of Rawdata obtained from the detection area PA that is due to the intensity of the light emitted from one point light source 22 located at the same coordinates as those of the detection area PA. In FIG. 13, “Br_Add” indicates a component of the output level of Rawdata obtained from the detection area PA that is due to the intensity of the light emitted from another point light source 22 located at coordinates adjacent to those of the detection area PA. As described with reference to FIG. 10, the output level that appears as “Br_Add” can vary depending on the number of the adjacent detection areas PA. However, FIG. 13 only schematically illustrates the generation of “Br_Add” and does not illustrate differences depending on the number of the adjacent detection areas PA. The following description assumes that “Br_Add” in FIG. 13 illustrates the component of the output in the detection areas PA at (X, Y)=(1, 1), (1, 3), (3, 1), and (3, 3). That is, “Br_Add” in each of the detection areas PA at (X, Y)=(1, 2), (2, 1), (2, 2), (2, 3), and (3, 2) is stronger than that illustrated in FIG. 13.

The output of the optical sensor WA is limited by the dynamic range. The dynamic range herein is the range of output between the upper and lower limits of the output of the optical sensor WA. That is, the output of the optical sensor WA can neither exceed the upper limit nor fall below the lower limit. FIG. 13 illustrates the upper limit of the output of the optical sensor WA as an upper limit DR.

In spite of the limitation by the dynamic range, such as the upper limit DR, the intensity of the light from the point light source 22 can be an intensity corresponding to an output exceeding the upper limit of the output of the optical sensor WA. For example, the point light source 22 in the first state St_1 described with reference to FIG. 12 emits light having an intensity corresponding to the luminance of “Br_Ma”. Light at an intensity corresponding to the luminance of “Br_Ma” is strong enough to produce an output of the optical sensor WA that exceeds the upper limit DR, as illustrated in FIG. 13.

In FIG. 13, not only light having an intensity corresponding to the luminance of “Br_Ma”, but also light having an intensity corresponding to the luminance of each of “Br_(Ma−1)” and “Br_(Ma−2)” have strength strong enough to produce an output of the optical sensor WA that exceeds the upper limit DR. “Br_(Ma−1)” corresponds to light that is weaker than “Br_Ma”, stronger than “Br_(Ma−2)”, and strong enough to produce an output of the optical sensor WA that exceeds the upper limit DR. “Br_Ma” and “Br_(Ma−1)” exceed the upper limit DR with only “Br_Base”. “Br_(Ma−2)” corresponds to light that is weaker than “Br_Ma” and “Br_(Ma−1)” and strong enough to produce an output of the optical sensor WA that exceeds the upper limit DR. “Br_(Ma−2)” does not exceed the upper limit DR with only “Br_Base”, but exceeds the upper limit DR with the addition of “Br_Add”. In contrast, the output of the optical sensor WA reaches the maximum at the upper limit indicated by the upper limit DR. Therefore, whether the intensity of the light from the point light source 22 is “Br_Ma”, “Br_(Ma−1)”, or “Br_(Ma−2)”, the output of the optical sensor WA reaches the maximum at the upper limit DR, and no difference appears depending on the intensity of the light from the point light source 22. Thus, the term “saturation of output” refers to the state in which the output of the optical sensor WA reaches the maximum at the upper limit DR and no difference appears depending on the intensity of the light from the point light source 22.

In the state in which the saturation of output occurs, the presence or absence of colonies may not appear in the output of the optical sensor WA. As described above, whether colonies have been formed is determined based on the bright/dark difference caused by the difference in degree of transmission of light between the culture medium with no colonies and the culture medium with colonies. However, if the saturation of output occurs, the bright/dark difference may fall within a range exceeding the upper limit DR, and the output of the optical sensor WA may not reflect the bright/dark difference. Thus, the effect of the formation of colonies may not appear in the image obtained in the state in which the saturation of output occurs. Such a situation is undesirable for determining whether colonies have been formed.

Therefore, in the embodiment, control is performed to turn on the point light source 22 at the luminance at which the output of the optical sensor WA is lower than the upper limit DR. Specifically, automatic luminance adjustment is performed. In the automatic luminance adjustment, a data acquisition process and a first luminance check process are performed. The data acquisition process is a process to acquire the image data at predetermined luminance of the point light source 22. The first luminance check process is a process to reduce the luminance of the point light source 22 at the same coordinates as those of the detection area PA that has produced the output exceeding a threshold Tr1, if the intensity of the output of the detection area PA (Rawdata) included in the image data obtained in the latest data acquisition process exceeds the threshold Tr1. In the automatic luminance adjustment, the data acquisition process and the first luminance check process are repeated while the detection area PA with the intensity of the output (Rawdata) exceeding the threshold Tr1 is present. The “predetermined luminance of the point light source 22” in the data acquisition process performed after the first luminance check process is the luminance of the point light source 22 after being lowered by the latest first luminance check process. In the embodiment, the “predetermined luminance of the point light source 22” in the first data acquisition process before the first luminance check process is performed is the highest luminance, that is, the luminance of “Br_Ma”. The luminance of “Br_Ma” may be the highest luminance in the technical specification that can be achieved by the point light source 22, as described above, or it may be the highest luminance within the “adjustable luminance range of the point light source 22” set in advance.

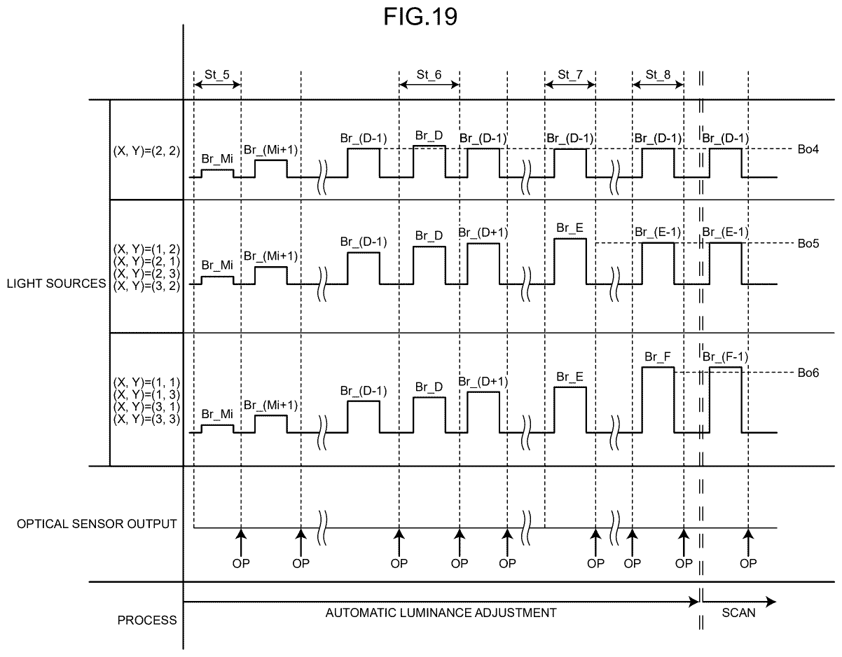

FIG. 14 is a timing diagram schematically illustrating a process of gradual reduction of the luminance levels of the point light sources 22 by the automatic luminance adjustment performed in the embodiment. In FIG. 14, the luminance of the point light source 22 that is lowered as the automatic luminance adjustment progresses, is schematically illustrated as the height of a rising edge of a rectangular wave. In FIG. 14 and in FIG. 19 to be explained later, an output OP in “output of optical sensor” in “automatic luminance adjustment” of “process” indicates the output of the optical sensor WA that is generated in the data acquisition process.

Explaining with reference to FIGS. 13 and 14, the image obtained by the point light source 22 lit at the luminance of “Br_Ma”, that is, the output (Rawdata) of the detection area PA, exceeds the threshold Tr1. Therefore, the first luminance check process reduces the luminance of the point light source 22 from “Br_Ma” to “Br_(Ma−1)”. However, Rawdata obtained by the point light source 22 lit at the luminance of “Br_(Ma−1)” also exceeds the threshold Tr1. Therefore, the first luminance check process reduces the luminance of the point light source 22 from “Br_(Ma−1)” to “Br_(Ma−2)”. Thus, in the embodiment, the automatic luminance adjustment gradually reduces the luminance of the point light source 22.

The threshold Tr1 is a predetermined threshold for outputs of the detection area PA. The threshold Tr1 is lower than the upper limit DR. In addition to being lower than the upper limit DR, the threshold Tr1 is preferably determined by taking into account at least one of the following first and second conditions, and more preferably by taking into account both of the first and second conditions. The first condition is that a margin of an output level is given that takes into account variations in brightness/darkness of light due to various conditions, such as the condition where the state of the culture medium is not perfectly uniform. That is, the first condition is to have a margin of output over the dynamic range such that the setting of the output level based on the threshold Tr1 is suitable for an assumed operation of the detection device 1, even in the presence of such variations in brightness/darkness. The second condition is to allow the output level to be high enough to be as close to the upper limit DR as possible within the dynamic range. With these conditions, a better image of the object to be detected 200 can be obtained in a brighter environment after the automatic luminance adjustment, making it easier to clarify the bright/dark difference in the image to be used for determining whether colonies have been formed.

A second state St_2 illustrated in FIG. 12 is a state after the first state St_1 in the automatic luminance adjustment, and is a state in which all the point light sources 22 are lit at lower luminance than that in the first state St_1. In the second state St_2 in FIGS. 12 and 14, the intensity of light corresponding to the luminance “Br_A” represents the luminance of the point light source 22. As described above, “Br_Add” in FIG. 13 illustrates the component of the output in the detection areas PA at (X, Y)=(1, 1), (1, 3), (3, 1), and (3, 3). As illustrated in FIG. 13, Rawdata obtained by the point light source 22 lit at luminance of “Br_A” falls below the threshold Tr1. Rawdata obtained from the detection area PA by the light of the point light source 22 lit at luminance of “Br_(A+1)” that is stronger than “Br_A” by one step exceeds the threshold Tr1. Thus, the first luminance check process is applied to all the point light sources 22 in the light-emitting area LA illustrated in FIG. 8 until the luminance of the point light source 22 is lowered from “Br_(A+1)” to “Br_A”. Then, in each of the detection areas PA at (X, Y)=(1, 1), (1, 3), (3, 1), and (3, 3), Rawdata obtained by the point light source 22 lit at the luminance of “Br_A” falls below the threshold Tr1. As a result, the luminance of each of the point light sources 22 located at the same coordinates as these detection areas PA stops decreasing. A dashed line Bo1 in FIG. 14 indicates that the luminance of each of the point light sources 22 at (X, Y)=(1, 1), (1, 3), (3, 1), and (3, 3) does not fall below “Br_A”.

As described with reference to FIG. 11, in the embodiment, each of the detection areas PA includes more than one optical sensor WA. Therefore, whether Rawdata of the detection area PA falls below the threshold Tr1 is determined by whether the average of the outputs of the respective optical sensors WA included in the detection area PA falls below the threshold Tr1. That is, the output of each of the detection areas PA is the average of the outputs of the optical sensors WA included in the detection area PA. The detection areas PA marked with “OK” in the “Image” row in FIG. 12 represent the detection areas PA where Rawdata falls below the threshold Tr1.

However, “Br_Add” in each of the detection areas PA at (X, Y)=(1, 2), (2, 1), (2, 2), (2, 3), and (3, 2) is stronger than that illustrated in FIG. 13. Therefore, in these detection areas PA, Rawdata obtained by the point light source 22 lit at the luminance of “Br_A” exceeds the threshold Tr1. Therefore, the luminance of each of the point light sources 22 located at the same coordinates as these detection areas PA is further reduced from “Br_A” to “Br_(A−1)” by the first check process. The detection areas PA marked with “NG1” in the “Image” row in FIG. 12 represent the detection areas PA where Rawdata exceeds the threshold Tr1.

Thus, the threshold Tr1 serves as a threshold for determining the output of each of the detection areas PA. Information indicating a threshold such as the threshold Tr1 is preset in the control circuit 30. Specifically, such information is stored in a register provided in the control circuit 30, for example. The first luminance check process included in the automatic luminance adjustment corresponds to a process to set the luminance of the point light source 22 after the luminance change as the adjusted luminance if predetermined conditions are satisfied. The predetermined conditions are that one of two output levels of the detection area PA obtained before and after the luminance change of the point light source 22 is higher than the threshold Tr1 and that the other of the two output levels is lower than the threshold Tr1. The term “before and after the luminance change” refers, for example, to the luminance change from “Br_(A+1)” to “Br_A” described above. Such a process is performed individually for each combination of the point light source 22 and the detection area PA corresponding to each other. The term the “combination of the point light source 22 and the detection area PA corresponding to each other” refers, for example, to a combination of the point light source 22 and the detection area PA that have the same coordinates that can be expressed in the form of (X, Y)=(α, β) in the embodiment.

A third state St 3 illustrated in FIG. 12 is a state after the second state St_2 in the automatic luminance adjustment, and is a state in which the point light sources 22 at (X, Y)=(1, 2), (2, 1), (2, 2), (2, 3), and (3, 2) are lit at lower luminance than that in the second state St_2. In the third state St 3 in FIGS. 12 and 14, the intensity of light corresponding to the luminance “Br_B” represents the luminance of each of the point light sources 22 at (X, Y)=(1, 2), (2, 1), (2, 2), (2, 3), and (3, 2). In the detection areas PA at (X, Y)=(1, 2), (2, 1), (2, 3), and (3, 2), Rawdata obtained from each of the detection areas PA by the light of the point light sources 22 lit at the luminance of “Br_B” falls below the threshold Tr1. As a result, as illustrated in FIG. 14, the luminance of each of the point light sources 22 located at the same coordinates as these detection areas PA stops decreasing. A dashed line Bo2 in FIG. 14 indicates that the luminance of each of the point light sources 22 at (X, Y)=(1, 2), (2, 1), (2, 3), and (3, 2) does not fall below “Br_B”.

However, “Br_Add” in the detection area PA at (X, Y)=(2, 2) is stronger than “Br_Add” in the detection areas PA at (X, Y)=(1, 2), (2, 1), (2, 3), and (3, 2). Therefore, in the detection area PA at (X, Y)=(2, 2), Rawdata obtained by the point light source 22 lit at the luminance of “Br_B” exceeds the threshold Tr1. Therefore, the luminance of the point light source 22 located at the same coordinates as those of the detection area PA at (X, Y)=(2, 2) is further reduced from “Br_B” to “Br_(B−1)” by the first check process.

A fourth state St 4 illustrated in FIG. 12 is a state after the third state St 3 in the automatic luminance adjustment, and is a state in which the point light source 22 at (X, Y)=(2, 2) is lit at lower luminance than that in the third state St 3. In the detection area PA at (X, Y)=(2, 2), Rawdata obtained from the detection area PA by the light of the point light source 22 lit at the luminance of “Br_C” falls below the threshold Tr1. As a result, as illustrated in FIG. 14, the luminance of each of the point light sources 22 located at the same coordinates as these detection areas PA stops decreasing. A dashed line Bo3 in FIG. 14 indicates that the luminance of the point light source 22 at (X, Y)=(2, 2) does not fall below “Br_C”.

The automatic luminance adjustment controls the luminance of each of the point light sources 22 so that Rawdata of all the detection areas PA falls below the threshold Tr1. As given above in the description of the automatic luminance adjustment, in the embodiment, the luminance of the point light source 22 at the start of the automatic luminance adjustment is the highest luminance (“Br_Ma”). The luminance of the point light source 22 is lower after the luminance is changed than before the luminance is changed.

The image data is acquired after the automatic luminance adjustment, whereby it is possible to acquire the image data with a clearer bright/dark difference caused by whether colonies have been formed. In FIG. 14 and in FIG. 19 to be explained later, such a process related to the acquisition of the image data performed after the automatic luminance adjustment is illustrated as the output OP of a “scan” in the “process”. In the embodiment, the automatic luminance adjustment and the acquisition of image data after the automatic luminance adjustment are individually performed for each of the first light source 22R, the second light source 22G, and the third light source 22B.

In the description with reference to FIGS. 12 and 14 and FIGS. 18 and 19 to be explained later, the luminance levels of the point light sources 22 at (X, Y)=(1, 1), (1, 3), (3, 1), and (3, 3) are simultaneously determined. In the description, the luminance levels of the point light sources 22 at (X, Y)=(1, 2), (2, 1), (2, 3), and (3, 2) are also simultaneously determined. These simultaneous determination of the luminance levels of the point light sources 22 are only exemplary, and do not limit the operation of the detection device 1. The luminance adjustment of the point light sources 22 is individually performed for each combination of the point light source 22 and the detection area PA corresponding to each other. Therefore, assume that the predetermined conditions are satisfied in some of the detection areas PA at (X, Y)=(1, 1), (1, 3), (3, 1), and (3, 3) and not satisfied in some other of the detection areas PA, due to, for example, individual differences between LEDs or variations in sensitivity of the optical sensors WA. Under these hypothetical conditions, the luminance of each of the point light sources 22 corresponding to the detection areas PA that satisfy the predetermined conditions is determined and remains unchanged, while the luminance of each of the point light sources 22 corresponding to the detection areas PA that do not satisfy the predetermined conditions is not determined. That is, the luminance of each of the point light sources 22 corresponding to the detection areas PA that do not satisfy the predetermined conditions continues to be changed in luminance by the first luminance check process included in the automatic luminance adjustment.

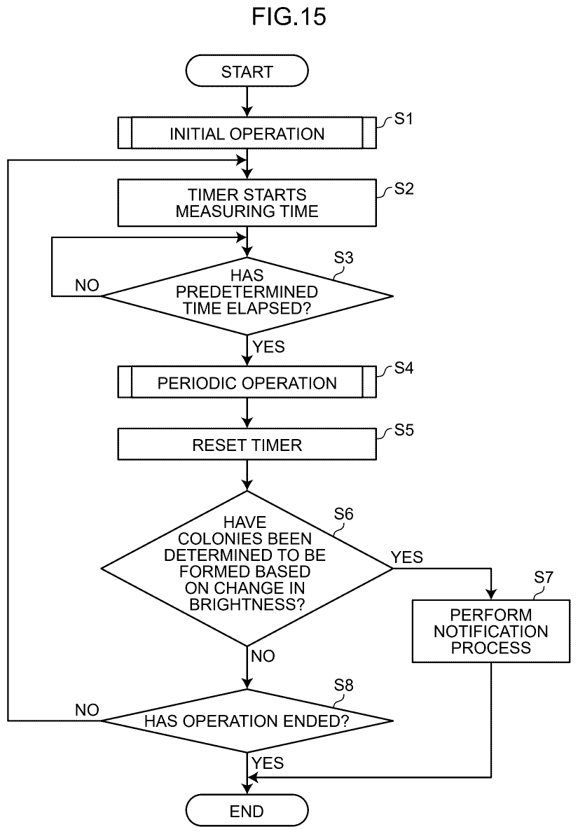

The following describes processing related to the operation of the detection device 1 with reference to flowcharts in FIGS. 15 to 17. Unless otherwise noted, in the embodiment, a process at each step illustrated in the flowcharts in FIGS. 15 to 17 is mainly performed by the control circuit 30.

FIG. 15 is a flowchart of processing related to the operations of the detection device 1. First, an initial operation is performed (Step S1). At the time of the initial process, the initial operation is performed immediately after the object to be detected 200 is placed on the detection device 1. That is, at the time of the initial process, no colonies have been formed on the culture medium.

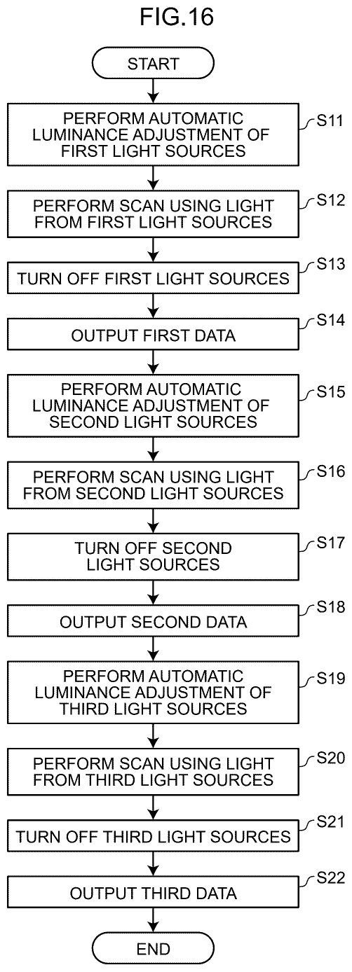

FIG. 16 is a flowchart of the initial process. First, the automatic luminance adjustment of the first light sources 22R is performed (Step S11). The automatic luminance adjustment in each of processes at Step S11 and at Steps S15 and S19 to be described later is the automatic luminance adjustment described with reference to FIGS. 12 to 14.

In the process at each of Step S11 and Steps S15 and S19 to be described later, the control circuit 30 operates the planar optical sensor 10 and the light source panel 20 to perform the automatic luminance adjustment.

The description of a process at Step S15 to be described later is obtained by replacing the first light sources 22R in the description of the process at Step S11 with the second light sources 22G. The description of a process at Step S19 to be described later is obtained by replacing the first light sources 22R in the description of the process at Step S11 with the third light sources 22B.

After the process at Step S11, a scan process using the light from the first light sources 22R is performed (Step S12). Specifically, the scan process is performed by the control circuit 30 operating the planar optical sensor 10 and the light source panel 20. In the process at Step S12, the light sources turned on by the operation of the light source panel 20 are the first light sources 22R. The second light sources 22G and the third light sources 22B are not turned on in the process at Step S12. As a result, the control circuit 30 obtains an image corresponding to the outputs of the optical sensors WA that have detected the light from the first light sources 22R transmitted through the object to be detected 200. At the completion of the process at Step S12, the first light sources 22R are turned off (Step S13).

In a process at Step S16 to be described later, the light sources to be turned on are not the first light sources 22R, but the second light sources 22G. In a process at Step S20 to be described later, the light sources to be turned on are not the first light sources 22R, but the third light sources 22B. After the processes at Steps S12 and S13, first data is output (Step S14). The first data is the image data obtained using the light from the first light sources 22R. Specifically, the control circuit 30 regards, as the first data, the image data reflecting the outputs of the optical sensors WA obtained in the process at Step S12.

After the process at Step S14, the automatic luminance adjustment of the second light sources 22G is performed (Step S15). After the process at Step S15, the scan process using the light from the second light sources 22G is performed (Step S16). Specifically, the scan process is performed by the control circuit 30 operating the planar optical sensor 10 and the light source panel 20. In the process at Step S16, the light sources turned on by the operation of the light source panel 20 are the second light sources 22G. The first light sources 22R and the third light sources 22B are not turned on in the process at Step S16. As a result, the control circuit 30 obtains an image corresponding to the outputs of the optical sensors WA that have detected the light from the second light sources 22G transmitted through the object to be detected 200. At the completion of the process at Step S16, the second light sources 22G are turned off (Step S17).

After the processes at Steps S16 and S17, second data is output (Step S18). The second data is the image data obtained using the light from the second light sources 22G. Specifically, the control circuit 30 regards, as the second data, the image data reflecting the outputs of the optical sensors WA obtained in the process at Step S16.

After the process at Step S18, the automatic luminance adjustment of the third light sources 22B is performed (Step S19). After the process at Step S19, the scan process using the light from the third light sources 22B is performed (Step S20). Specifically, the scan process is performed by the control circuit 30 operating the planar optical sensor 10 and the light source panel 20. In the process at Step S20, the light sources turned on by the operation of the light source panel 20 are the third light sources 22B. The first light sources 22R and the second light sources 22G are not turned on in the process at Step S20. As a result, the control circuit 30 obtains an image corresponding to the outputs of the optical sensors WA that have detected the light from the third light sources 22B transmitted through the object to be detected 200. At the completion of the process at Step S20, the third light sources 22B are turned off (Step S21).

After the processes at Steps S20 and S21, the third data is output (Step S22). The third data is the image data obtained using the light from the third light sources 22B. The control circuit 30 regards, as third data, the image data reflecting the outputs of the optical sensors WA obtained in the process of Step S20.

The initial operation ends with the completion of the process at completion of the first Step S22. As illustrated in FIG. 15, after the initial operation that is the process at Step S1, a timer starts measuring time (Step S2). The process at Step S2 may be performed, for example, by a timer circuit provided in the control circuit 30, by setting a variable that serves as a counter and updating the counter based on an operating clock of the control circuit 30, or by other methods.

After the start of measuring time by the process at Step S2, a check is made to determine whether a predetermined time has elapsed (Step S3). Until the predetermined time elapses, the control circuit 30 waits (No at Step S3), without performing the next process. The predetermined time is five minutes, for example, but is not limited thereto. The predetermined time may be determined as appropriate according to a cycle (time interval) at which determination of the formation of colonies is to be made. When the predetermined time has elapsed after the process at Step S2 (Yes at Step S3), a periodic operation is performed (Step S4).

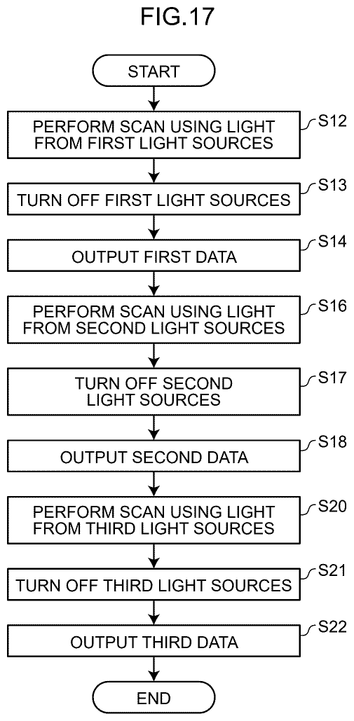

FIG. 17 is a flowchart of the periodic operation. The periodic operation is an operation in which the processes at Steps S11, S15, and S19 are omitted from the processes included in the initial operation described with reference to FIG. 16. In the periodic operation, the processes are performed in the following order: Step S12, Step S13, Step S14, Step S16, Step S17, Step S18, Step S20, Step S21, and Step S22.

The first light sources 22R, the second light sources 22G, and the third light sources 22B are turned on at different times. While one group of a group of the first light sources 22R, a group of the second light sources 22G, and a group of the third light sources 22B is on, the other two groups are not on. These light sources are periodically turned on in the order of the first light sources 22R, the second light sources 22G, and the third light sources 22B. These operations are indicated by the processes at Steps S12, S13, S16, S17, S20, and S21 in the initial operation and the periodic operation.

The luminance of the first light sources 22R that are turned on in the periodic operation is the luminance adjusted by the automatic luminance adjustment by the process at Step S11 in the initial operation. The luminance of the second light sources 22G that are turned on in the periodic operation is the luminance adjusted by the automatic luminance adjustment by the process at Step S15 in the initial operation. The luminance of the third light sources 22B that are turned on in the periodic operation is the luminance adjusted by the automatic luminance adjustment by the process at Step S19 in the initial operation.

The periodic operation ends with the completion of the process at Step S22 at the second and subsequent times. As illustrated in FIG. 15, after the periodic operation that is the process at Step S4, the timer is reset (Step S5). That is, the timer that started measuring time at Step S2 is reset in the process at Step S5.

The control circuit 30 determines whether colonies have been formed based on a change in brightness between the data obtained in the initial operation and the data obtained in the periodic operation (Step S6). Specifically, the control circuit 30 compares t-th data obtained in the initial operation with the t-th data obtained in the periodic operation. If a dark area not included in the t-th data obtained in the initial operation is included in the t-th data obtained in the periodic operation, the control circuit 30 determines that the dark area is due to colonies. t in the t-th data is 1, 2, or 3. To illustrate a case where t is 1, the control circuit 30 compares the first data obtained in the initial operation with the first data obtained in the periodic operation. If the dark area not included in the first data obtained in the initial operation is included in the first data obtained in the periodic operation, the control circuit 30 determines that the dark area is due to colonies. The same interpretation only needs to be made also for a case where t=2 or t=3. The control circuit 30 individually determines the case where t=1, the case where t=2, and the case where t=3. The time point at which the size of the dark area has become large enough to be regarded as the colonies is determined in advance and can be changed as appropriate depending on the size of the colonies at which a notification is to be made by a notification process to be described later.

In the embodiment, if a dark area considered to be a colony is formed in one or more of a case where t=1, a case where t=2, and a case where t=3, it is regarded that a colony is determined to have been formed in the process at Step S6. However, specific conditions for such determination are not limited to this condition. If a dark area considered to be a colony is formed in two or more or all three of the case where t=1, the case where t=2, and the case where t=3, a colony may be determined to have been formed in the process at Step S6. The process at Step S6 corresponds to the determination process to determine whether a colony is formed based on a comparison among a plurality of images obtained at different times.

If the process at Step S6 determines that a colony has been formed (Yes at Step S6), the notification process is performed (Step S7). In the notification process, a predetermined notification method is used to perform the notification. In the embodiment, the notification process is performed to send electronic mail indicating the formation of the colony to an electronic mail address of a manager of the object to be detected 200. The electronic mail and a text to be sent via the electronic mail are set in advance. In the embodiment, for example, the control circuit 30 serves as a sender of the electronic mail, but is not limited to this method. As another example, the control circuit 30 may output, to an external information processing device, a signal that serves as an instruction for the external information processing device to send the electronic mail, or may use other methods. The form of the notification performed in the notification process is not limited to the sending of the electronic mail. For example, a voice output device such as a speaker may be operated to output predetermined “voice to notify that a colony has been formed” or other forms of notification may be used. The process at Step S7 corresponds to a process to make an output indicating that a colony has been formed when the colony is determined to have been formed.

If the process at Step S6 determines that no colonies have been formed (No at Step S6), the process at Step S2 is re-performed unless the detection device 1 has ended operating (No at Step S8). That is, the timer measures time again, and the periodic operation, the resetting of the timer, and determination of whether a colony has been formed are performed each time the predetermined time elapses. If the detection device 1 has ended operating in the process at Step S8 (Yes at Step S8) or after the process at Step S7 is performed, the processing related to the operations of the detection device 1 ends.

As described above, according to the embodiment, the detection device 1 includes the light source part (light source panel 20) in which the point light sources (point light sources 22) that emit light are two-dimensionally arranged, the planar optical sensor (planar optical sensor 10) in which the optical sensors (optical sensors WA) that detect the light from the light source part are two-dimensionally arranged, the object placement member (member 60) provided so that the object to be detected (object to be detected 200) can be placed between the light source and the planar optical sensor, and the control circuit (control circuit 30) that controls operations of the light source part and the planar optical sensor. The planar optical sensor has a plurality of detection areas (detection areas PA) corresponding to the arrangement of the point light sources. Each of the detection areas includes a plurality of the optical sensors. The control circuit performs luminance adjustment (such as the automatic luminance adjustment described above) of the point light sources. In the luminance adjustment, when the predetermined conditions are satisfied, the process, in which the luminance of the point light source before or after (for example, after) the luminance change is set as the luminance of the point light source after the adjustment, is individually performed for each combination of the point light source and the detection area corresponding to each other. The predetermined conditions are that one of two output levels of the detection area obtained before and after changing the luminance of the point light source is higher than the threshold for determining the output of each of the detection areas, and that the other of the two output levels of the detection area is lower than the threshold. The threshold is the threshold Tr1, for example. This adjustment adjusts the luminance of the point light source to luminance that is neither too bright nor too dark in terms of the detection of light by the optical sensors. Thus, the embodiment allows better detection of the colonies.

The luminance of the point light source (point light source 22) at the start of the luminance adjustment of the point light source (point light source 22) by the automatic luminance adjustment described above is the highest luminance (“Br_Ma”). In the luminance adjustment of the point light source, the luminance of the point light source is lower after the luminance is changed than before the luminance is changed, and the process is performed in which the luminance of the point light source is changed when the predetermined conditions are satisfied and the luminance after the change is set as the adjusted luminance. Such a process is individually performed for each combination of the point light source and the detection area (detection area PA) corresponding to each other. This process allows the luminance of the point light source to be more accurately adjusted to luminance that is not too bright in terms of the detection of light by the optical sensors.

The point light source (point light source 22) includes the first light source (first light source 22R) that emits the red light, the second light source (second light source 22G) that emits the green light, and the third light source (third light source 22B) that emits the blue light. The luminance adjustment of the point light source by the automatic luminance adjustment described above is individually performed for the first light source, the second light source, and the third light source. Thus, the outputs of the planar optical sensor (planar optical sensor 10) corresponding to three colors of light that constitute what is called red-green-blue (RGB) image data are obtained. Therefore, optical effects produced by the colonies in the culture medium can be acquired more reliably.

The optical member (member 60) is provided between the object to be detected (object to be detected 200) and the planar optical sensor (planar optical sensor 10), and the optical member (member 60) includes any one of the plate-shaped louver, the cylindrical opening, and the microlens. With this configuration, the area through which light to be detected by each of the optical sensors (optical sensors WA) passes can be easily limited to an area facing the optical sensor.

### Modification

The following describes a modification of the embodiment having a partially different configuration from the embodiment described above, with reference to FIGS. 18 and 19. In the description of the modification, the same components as those in the embodiment are denoted by the same reference numerals and will not be described again.

FIG. 18 illustrates schematic views illustrating another example of the process to sequentially determine the intensity of the light emitted from each of the point light sources 22. In the modification, the first luminance check process in the embodiment is replaced with a second luminance check process. In the second luminance check process, if the intensity of the output of the detection area PA (Rawdata) included in the image data obtained in the latest data acquisition process is below a predetermined threshold, the luminance of the point light source 22 at the same coordinates as those of the detection area PA that has produced the output below the threshold is increased. In the automatic luminance adjustment of the modification, the data acquisition process and the second luminance check process are repeated while the detection area PA with the intensity of the output (Rawdata) less than the threshold is present. The “predetermined luminance of the point light source 22” in the data acquisition process performed after the second luminance check process is the luminance of the point light source 22 after being increased by the latest second luminance check process. In the modification, the “predetermined luminance of the point light source 22” in the first data acquisition process before the second luminance check process is performed is the luminance corresponding to the lowest luminance. In FIG. 18, “Br_Mi” indicates the lowest luminance. The point light source 22 in a fifth state St_5 illustrated in FIG. 18 emits light at an intensity corresponding to the luminance of “Br_Mi”.

Furthermore, in the automatic luminance adjustment according to the modification, a luminance restoration process is performed. In the luminance restoration process, if the intensity of the output (Rawdata) of the detection area PA included in the image data obtained in the latest data acquisition process exceeds the predetermined threshold of the modification, the luminance of the point light source 22 at the same coordinates as those of the detection area PA that has produced the output exceeding the threshold is reduced. The degree of reduction of the luminance of the point light source 22 in the luminance restoration process is a degree of “canceling the degree of a single luminance increase lastly applied to the luminance of the point light source 22”.

FIG. 19 is a timing diagram schematically illustrating a process of gradual increase of the luminance levels of the point light sources 22 by the automatic luminance adjustment performed in the modification. In FIG. 19, the luminance of the point light source 22 that is increased as the automatic luminance adjustment progresses is schematically indicated by the height of a rising edge of a rectangular wave.

Explaining with reference to FIGS. 18 and 19, the image obtained by the point light sources 22 lit at the luminance of “Br_Mi”, that is, the output (Rawdata) of each of the detection areas PA, falls below the threshold of the modification. Therefore, the second luminance check process increases the luminance of each point light source 22 from “Br_Mi” to “Br_(Ma+1)”. However, Rawdata obtained by the point light source 22 lit at the luminance of “Br_(Ma+1)” also falls below the threshold. Therefore, the second luminance check process increases the luminance of the point light source 22 again. Thus, in the modification, the automatic luminance adjustment gradually increases the luminance of the point light source 22. The threshold is lower than the upper limit DR as with the threshold Tr1.

A sixth state St_6 illustrated in FIG. 18 is a state after the fifth state St_5, and is a state in which the point light sources 22 are all lit at higher luminance than in the fifth state St_5. In the sixth state St_6 in FIGS. 18 and 19, the luminance of each of the point light sources 22 is illustrated at a light intensity corresponding to the luminance of “Br_D”. In the detection area PA at (X, Y)=(2, 2), Rawdata obtained from the detection area PA by the light of the point light source 22 lit at the luminance of “Br_D” exceeds the threshold of the modification. As a result, the luminance of the point light source 22 located at the same coordinates as the detection area PA at (X, Y)=(2, 2) stops increasing, as illustrated in FIG. 19. The luminance restoration process described above restores the luminance “Br_(D−1)” of the point light source 22 at (X, Y)=(2, 2), from “Br_D”. A dashed line Bo4 in FIG. 19 indicates that the luminance of the point light source 22 at (X, Y)=(2, 2) does not exceed “Br_(D−1)” after being restored by the luminance restoration process. The detection areas PA marked with “OK” in the “Image” row in FIG. 18 represent the detection areas PA where Rawdata exceeds the threshold.

Thus, the second luminance check process included in the automatic luminance adjustment of the modification corresponds to a process to set the luminance of the point light source 22 before the luminance change as the adjusted luminance if the predetermined conditions are satisfied. The term “before and after the luminance change” refers, for example, to the luminance change from “Br_(D−1)” to “Br_D” described above.

In contrast, in each of the detection areas PA at (X, Y)=(1, 1), (1, 2), (1, 3), (2, 1), (2, 3), (3, 1), (3, 2), and (3, 3), Rawdata obtained by the point light source 22 lit at the luminance of “Br_D” falls below the threshold of the modification. Therefore, the luminance of each of the point light sources 22 located at the same coordinates as these detection areas PA is further increased from “Br_D” to “Br_(D+1)” by the second check process. The detection areas PA marked with “NG2” in the “Image” row in FIG. 18 represent the detection areas PA where Rawdata falls below the threshold.

A seventh state St_7 illustrated in FIG. 18 is a state after the sixth state St_6. In the seventh state, the point light sources 22 at (X, Y)=(1, 1), (1, 2), (1, 3), (2, 1), (2, 3), (3, 1), (3, 2), and (3, 3) are lit at higher luminance than that of the sixth state St_6. In the seventh state St_7 in FIGS. 18 and 19, the intensity of light corresponding to the luminance “Br_E” represents the luminance of each of the point light sources 22 at (X, Y)=(1, 1), (1, 2), (1, 3), (2, 1), (2, 3), (3, 1), (3, 2), and (3, 3). In each of the detection areas PA at (X, Y)=(1, 2), (2, 1), (2, 3), and (3, 2), Rawdata obtained from the detection area PA by the light of the point light source 22 lit at the luminance of “Br_E” exceeds the threshold of the modification. As a result, the luminance of each of the point light sources 22 located at the same coordinates as these detection areas PA stops increasing, as illustrated in FIG. 19. The luminance restoration process described above restores the luminance “Br_(E−1)” of the point light sources 22 at (X, Y)=(1, 2), (2, 1), (2, 3), and (3, 2), from “Br_E”. A dashed line Bo5 in FIG. 19 indicates that the luminance of each of the point light sources 22 at (X, Y)=(1, 2), (2, 1), (2, 3), and (3, 2) does not exceed “Br_(E−1)”.

In contrast, in each of the detection areas PA at (X, Y)=(1, 1), (1, 3), (3, 1), and (3, 3), Rawdata obtained by the point light source 22 lit at the luminance of “Br_E” falls below the threshold of the modification. Therefore, the luminance of each of the point light sources 22 located at the same coordinates as these detection areas PA is further increased from “Br_E” to “Br_(E+1)” by the second check process.

An eighth state St_8 illustrated in FIG. 18 is a state after the seventh state St_7, and is a state in which the point light sources 22 at (X, Y)=(1, 1), (1, 3), (3, 1), and (3, 3) are lit at higher luminance than that in the seventh state St_7. In each of the detection areas PA at (X, Y)=(1, 1), (1, 3), (3, 1), and (3, 3), Rawdata obtained from the detection area PA by the light of the point light source 22 lit at the luminance of “Br_F” exceeds the threshold of the modification. As a result, the luminance of each of the point light sources 22 located at the same coordinates as these detection areas PA stops increasing, as illustrated in FIG. 19. The luminance restoration process described above restores the luminance “Br_(F−1)” of the point light sources 22 at (X, Y)=(1, 1), (1, 3), (3, 1), and (3, 3), from “Br_F”. A dashed line Bo6 in FIG. 19 indicates that the luminance of each of the point light sources 22 at (X, Y)=(1, 1), (1, 3), (3, 1), and (3, 3) does not exceed “Br_(F−1)”.

The automatic luminance adjustment of the modification controls the luminance of each of the point light sources 22 so that Rawdata of all the detection areas PA exceeds the threshold of the modification. The image data is acquired after the automatic luminance adjustment, whereby it is possible to acquire the image data with a clearer bright/dark difference caused by whether colonies have been formed. In the modification, even after, for example, the luminance of the point light source 22 at coordinates (2, 2) no longer increases from “Br_D”, the luminance of the point light source 22 at coordinates adjacent to those coordinates increases, which slightly further increases the output of the detection area PA at those coordinates. Considering the effect of such an increase in the luminance of the point light source 22 at the adjacent coordinates, the threshold of the modification is determined in such a manner as not to cause saturation of the output due to such an increase in the luminance. Except for the matters noted above, the modification is the same as the embodiment.

In the modification, at the start of the luminance adjustment of the point light source (point light source 22) by the automatic luminance adjustment described above, the luminance of the point light source is the lowest luminance (“Br_Mi”). In the luminance adjustment of the point light source, the luminance of the point light source is higher after the luminance is changed than before the luminance is changed, and the process, in which the luminance of the point light source is changed when the predetermined conditions are satisfied and the luminance before the change is set as the adjusted luminance. Such a process is individually performed for each combination of the point light source and the detection area (detection area PA) corresponding to each other. This process allows the luminance of the point light source to be more accurately adjusted to luminance that is not too dark in terms of the detection of light by the optical sensors.

In the embodiment, the point light source 22 including the first light source 22R, the second light source 22G, and the third light source 22B is employed as the light source, but the light source that can be employed in the embodiment according to the present disclosure is not limited to this light source. For example, light sources corresponding to light in four or more colors of light may be employed, or light sources corresponding to one or two colors of light may be employed. Light in combined colors may also be used by simultaneously turning on some or all of a plurality of types of light sources that emit light in different colors. For example, when the first light sources 22R, the second light sources 22G, and the third light sources 22B are simultaneously turned on, white light is obtained.

The vertical positional relation between the planar optical sensor 10 and the light source panel 20 is not limited to the example illustrated in FIG. 7, and may be reversed from the relation illustrated in FIG. 7. In the embodiment, the member 60 is an optical member provided between the object to be detected 200 and the planar optical sensor 10 and is also the object placement member, but this configuration is only an example and is not limited to thereto. For example, a configuration that serves as such an optical member and a configuration that serves as the object placement member may be provided separately.

Other operational advantages accruing from the aspects described in the present embodiment that are obvious from the description herein, or that are conceivable as appropriate by those skilled in the art will naturally be understood as accruing from the present disclosure.

What is claimed is:

1. A detection device comprising:
a light source part in which a plurality of point light sources configured to emit light are two-dimensionally arranged;
a planar optical sensor in which a plurality of optical sensors configured to detect the light from the light source part are two-dimensionally arranged;
an object placement member provided to allow an object to be detected to be placed between the light source part and the planar optical sensor; and
a control circuit configured to control operations of the light source part and the planar optical sensor, wherein
the planar optical sensor has a plurality of detection areas corresponding to an arrangement of the point light sources,
each of the detection areas comprises more than one of the optical sensors,
the control circuit is configured to perform luminance adjustment of the point light sources, and
in the luminance adjustment:
when predetermined conditions are satisfied, a process, in which luminance of the point light source before or after luminance change is set as adjusted luminance of the point light source, is individually performed for each combination of the point light sources and the detection areas corresponding to each other; and
the predetermined conditions are that one of two output levels of each of the detection areas obtained before and after changing the luminance of each of the point light sources is higher than a threshold for determining an output of the detection area, and that another of the two output levels of the detection area is lower than the threshold.

2. The detection device according to claim 1, wherein
the luminance of each of the point light sources at start of the luminance adjustment is highest luminance, and
in the luminance adjustment:
the luminance of the point light source is lower after the luminance is changed than before the luminance is changed, and
the process, in which the luminance of the point light source is changed when the predetermined conditions are satisfied and the luminance after the change is set as the adjusted luminance of the point light source, is individually performed for each combination of the point light sources and the detection areas corresponding to each other.

3. The detection device according to claim 1, wherein
the luminance of each of the point light sources at start of the luminance adjustment is lowest luminance, and
in the luminance adjustment:
the luminance of the point light source is higher after the luminance is changed than before the luminance is changed, and
the process, in which the luminance of the point light source is changed when the predetermined conditions are satisfied and the luminance before the change is set as the adjusted luminance of the point light source, is individually performed for each combination of the point light sources and the detection areas corresponding to each other.

4. The detection device according to claim 1, wherein
the point light sources comprise first light sources configured to emit red light, second light sources configured to emit green light, and third light sources configured to emit blue light, and
the luminance adjustment is individually performed for the first light source, the second light source, and the third light source.

5. The detection device according to claim 1, comprising an optical member provided between the object to be detected and the planar optical sensor, wherein
the optical member comprises any one of a plate-shaped louver, a cylindrical opening, and a microlens.
